(19) United States
(12) Patent Application Publication
(10) Pub. No.: US 2025/0384293 A1
(43) Pub. Date: Dec. 18, 2025

(54) METHOD OF EMOTION RECOGNITION IN CROSS-SUBJECT EEG SIGNALS

(71) Applicant: DALIAN UNIVERSITY, Dalian (CN)

(72) Inventors: Xiuli DU, Dalian (CN); Xinyue WANG, Dalian (CN); Fang WANG, Dalian (CN); Yana LV, Dalian (CN); Shaoming QIU, Dalian (CN); Lingyan HU, Dalian (CN)

(21) Appl. No.: 19/266,060

(22) Filed: Jul. 10, 2025

Related U.S. Application Data
(63) Continuation of application No. PCT/CN2024/143186, filed on Dec. 27, 2024.

(30) Foreign Application Priority Data
Jun. 13, 2024 (CN) 202410761424.3

(51) Int. Cl. G06N 3/096; G06N 3/0442

(52) U.S. Cl. CPC G06N 3/096; G06N 3/0442

(57) ABSTRACT

A method of emotion recognition in cross-subject EEG signals, belonging to technical field of deep learning, includes the following steps: S1, constructing the extracted DE features into positive and negative samples by using a positive and negative sample generator; S2, sending the DE features of an anchor and the positive and negative samples into the encoder for coding, mapping the DE features to a latent space, performing regression prediction on the encoded anchor samples in the latent space by using an autoregressive model, training the encoder by using a probability supervision contrastive loss function; and S3, connecting the trained encoder to the classifier for fine tuning, and training the classifier through the cross entropy loss function; in this process, the encoder does not perform gradient propagation to complete cross-subject emotion recognition.

### CROSS-REFERENCE TO RELATED APPLICATION

This patent application n a continuation of International Application No. PCT/CN2024/143186, filed on Dec. 27, 2024, and claims the benefit and priority of Chinese Patent Application No. 202410761424.3, entitled “METHOD OF EMOTION RECOGNITION IN CROSS-SUBJECT EEG SIGNALS” filed with the China National Intellectual Property Administration on Jun. 13, 2024, the disclosure of which is incorporated by reference herein in its entirety as part of the present disclosure.

### TECHNICAL FIELD

The present disclosure belongs to the technical field of deep learning, and specifically relates to a method of emotion recognition in cross-subject EEG signals.

### BACKGROUND

Emotion recognition is a key technology for achieving advanced human-computer interaction, which is widely used in fields such as psychology, artificial intelligence, medical treatment, entertainment services, etc., and helps to improve the humanization level of machines and enhance the experience of human-computer interaction. Compared with non-physiological signals such as facial expression, body posture and voice, the Electroencephalogram (EEG) signal directly reflects the activity of the brain, is not easily influenced by the individual's subjective consciousness and intention, and has higher temporal resolution, so it can provide objective and real emotional state information. The recognition rate of the EEG signal is usually high, which can accurately distinguish different emotional states. Therefore, the emotion recognition based on EEG signals is of great significance for the development of human-computer interaction. With the development of the deep learning, more and more classification and recognition models of deep learning have been applied to EEG emotion recognition. The artificially designed features, such as Power Spectral Density (PSD), Differential Entropy (DE), or the encoded image features, such as time-frequency map, spectrogram and other input forms have been utilized by the deep learning, or the various advanced networks and learning frameworks combined with the deep learning, such as convolutional neural network, graph neural network, transformer based on attention mechanism, etc., which have achieved extremely high accuracy in the field of emotion recognition. At the same time, compared with the image features, the research shows that using the artificially designed DE features as the input of the deep model will achieve a more stable and higher recognition rate. With the continuous progress of EEG acquisition technology and signal processing technology, the emotion recognition using the EEG signal has made many widely recognized research results.

Traditional emotion recognition models usually require personalized training for each subject, which requires a large number of experiments and data annotation. In this case, the accuracy of intra-subject emotion recognition has reached more than 97% since 2022. However, in practical applications, we often face new subjects, whose emotional features and expressions may be different from those of the subjects in the training set. This is because in the same task, different subjects have different skull shapes and different sensitivity to stimuli, which leads to individual differences in physiological activities among subjects. Therefore, it is more challenging to establish a common recognition method for all subjects and improve the accuracy of cross-subject emotion recognition than to be independent. Traditional machine learning algorithms usually rely on the assumption that training data and test data are independent and identically distributed when dealing with cross-subject tasks. However, this assumption often leads to a sharp decline in the performance of trained traditional classifiers when performing cross-subject tasks. In the past two years, the cross-subject emotion recognition is mainly solved by the deep learning methods, mainly including transfer learning, which includes Domain Adaptation (DA) and Domain Generalization (DG). The DA takes the samples of the training set as the source domain and the samples of the test set as the target domain. The model minimizes the data distribution difference between the two domains by transferring the knowledge obtained in the source domain to the target domain. Although the accuracy of the DA can be improved by 20% compared with machine learning, this model must measure the difference between the two domains through some samples of the test set, that is to say, the model needs some data of the test set when it is trained, so the model still needs to be retrained for those subjects who have never met in the network. Compared with the DA, the DG is also divided into two domains. Its purpose is to find domain-invariant features in the source domain, and it does not need to access the data of the test set. It also has outstanding performance in cross-subject tasks, so it has attracted more attention from researchers. In addition to transfer learning, Xin ke, Shen et al. first applied contrastive learning to the cross-subject emotion recognition. The method adopts the contrastive learning to maximize the similarity of the features of the positive sample pairs in the same emotional stimulus and minimize the similarity of the features of the negative sample pairs in different stimuli, with an accuracy rate of 86%, which surpassed the transfer learning developed for many years. It can be seen that contrastive learning has great development potential in the cross-subject emotion recognition. However, in this study, like most self-supervised contrastive learning, there are only one positive sample pair and fewer emotion categories, while the EEG-based emotion recognition is different from the recognition task in computer vision. Therefore, during training, a large number of pseudo-negative samples will be used as negative samples to push away anchor samples, affecting the final recognition accuracy. The above previous work shows that there are great differences in emotional expression among subjects, and there must be a constant representation among subjects, but it is a challenge to have a high recognition accuracy in the cross-subject case without accessing the test set data in the process of model training, so it is feasible and meaningful to explore the method of cross-subject emotion recognition.

### SUMMARY

In order to solve the above problems, the present disclosure provides a method of emotion recognition in cross-subject EEG signals, which includes the following steps.

S1, constructing the extracted DE features into positive and negative samples by using a positive and negative sample generator;

S2, sending the DE features of an anchor and the positive and negative samples into the encoder for coding, mapping the DE features of an anchor and the positive and negative samples to a latent space, performing regression prediction on the encoded anchor samples in the latent space by using an autoregressive model, training the encoder by using a supervision contrastive loss function, training the encoder to complete representation learning by narrowing the distance between positive sample pairs and widening the distance between negative sample pairs, and discarding the autoregressive model after the representation learning is completed; and

S3, connecting the trained encoder to the classifier for fine tuning, and training the classifier through the cross entropy loss function; in this process, the encoder does not perform gradient propagation to complete cross-subject emotion recognition.

Furthermore, in the constructed positive and negative samples, a strategy is set by combining the positive and negative samples of the supervised contrastive loss, the label information of the samples is included in the design of the positive and negative samples, and a mini batch generated by the positive and negative sample generator is used as the input of the contrastive learning encoder. Defining that I={+,−,× . . . } represents the set of emotions, taking the SEED dataset as an example, representing three types of emotions respectively: happy, sad and neutral, S={1,2,3 . . . n} represents the set of n subjects, all samples can be marked as

$${X}_{q}^{k} , {X}_{q}^{k} ∈ H ,$$

q_{∈S}, k_{∈I},

$${X}_{q}^{k} ∈ {R}^{C * D} ,$$

C represents the number of channels, D represents the feature dimension extracted within a certain time, and H represents all sample sets under this dataset.

In a batch, first determine the fixed emotion sample

$$f ∈ {X}_{1}^{+}$$

of the subject 1, and then take samples of subject 1 under the same emotion as positive samples in each experiment, that is,

$${f}^{+} ∈ {X}_{q}^{+} ( q ∈ S ) .$$

The number of f⁺ is n*p⁺, and p⁺ represents the number of experimental segments in the dataset that evoke +emotions. Take all samples of subjects with different emotions from the positive samples in each experiment as negative samples, that is,

$$f ∈ {X}_{q}^{k} ( q ∈ S , k ∈ I , k \neq + ) ,$$

wherein the number f is n*p^{k}(k∈I,k≠+).

In order to fully capture the features of samples in a batch, the mini batch is extended by taking 6 consecutive sample sequences for 2 seconds per sample. Definition: In the process of a fixed subject conducting an experiment, that is, the emotion caused by a certain stimulus, such as in the SEED dataset, wherein the average duration of the stimulus is 4 minutes and there are 3 types of emotion classifications, 20 anchor samples will be generated, with 4*60/(2*6)=20 anchor samples. Each anchor corresponds to N positive samples and 2N negative samples, and their set e={f,f⁺,f} is used as a batch. In the next batch, the anchors and positive and negative samples are reselected until all samples are used as anchors, and then the training of an epoch is completed.

Furthermore, a feature extraction network is constructed by a contrastive predictive coding design, so that the positive sample pairs are close to each other and the negative sample pairs are far away from each other.

First, a nonlinear encoder g_{enc} maps an input sequence x(t) to a latent representation sequence z(t)=g_{enc}(xₜ), and an autoregressive model gₐᵣ summarizes all z≤t in the latent space and predicts a latent representation c(t)=gₐᵣ(z(t)). In contrastive predictive coding learning, a residual structure is used as the encoder g_{enc} to avoid over-fitting, the anchor samples and the positive and negative samples enter the encoder in batches to obtain zₖ(t), the anchor samples enter an LSTM the autoregressive model gₐᵣ to obtain a prediction result c(t). The LSTM is added as the the autoregressive model gₐᵣ to improve the time resolution of features. In the prediction process, the network learns the underlying features of the anchor emotions, the prediction result c(t) is a feature representation with anchor emotions. The prediction result and the feature z₊(t) obtained by coding the positive sample form a positive sample pair; the prediction result and the feature z₋(t) formed with the negative sample coding is a negative sample pair; and finally the distance of the positive sample pair is narrowed and the distance of the negative sample pair is widened through the supervised contrastive loss function to complete the contrastive predictive coding.

The correct sample is distinguished from a set of noise samples by the Noise Contrastive Estimation (NCE) loss function, and the model is trained by maximizing the probability of the correct samples and minimizing the probability of the noise samples; in contrastive learning, the model is trained by comparing the positive sample and the negative sample, as shown in formula (1):

$${L}_{p} = - log \frac{exp ( m \cdot {p}_{+} / τ )}{{\sum }_{i = 0}^{K} exp ( m \cdot {p}_{i} / τ )} \qquad ( 1 )$$

Wherein, m [symbol] p is the representation vector obtained by the sample passing through the f(·) network, m·p₊ is the dot product similarity between the anchor and the positive sample, m·pᵢ is the dot product similarity between the anchor and other samples, K represents the number of negative samples, and τ is a temperature parameter.

In combination with the idea of contrastive predictive coding (CPC), the training of both the encoder and the the autoregressive model gₐᵣ is also included in this loss function, and both the encoder and the the autoregressive model gₐᵣ are trained to jointly optimize the loss based on NCE, as shown in formula (2):

$${L}_{N} = - log \frac{exp ( {c}_{h} \cdot {z}_{q} / τ )}{{\sum }_{a ▯ A ( h )} exp ( {c}_{h} \cdot {z}_{a} / τ )} \qquad ( 2 )$$

Wherein, cₕ is the predicted vector of the anchor sample h obtained through gₐᵣ(g_{enc}(xₜ)), z_{q} is the representation vector of the positive sample of the subject q obtained through g_{enc}(xₜ), A(h)=e\h, zₐ are the representation vectors of samples other than anchors in a batch obtained through g_{enc}(xₜ); Unlike CPC, which only considers samples from anchors as positive samples, using label information combined with CPC loss, each anchor can have multiple positive samples, that is, samples with the same label are positive samples, making contrastive learning suitable for fully supervised situations, as shown in the following formula (3):

$${L}^{sup} = {\sum }_{h ▯ H} {L}_{h} = {\sum }_{h ▯ H} \frac{- 1}{\vert q ( h ) \vert} {\sum }_{q ▯ S} log \frac{exp ( {c}_{h} \cdot {z}_{q} / τ )}{{\sum }_{a ▯ A ( h )} exp ( {c}_{h} \cdot {z}_{a} / τ )} \qquad ( 3 )$$

q(h) represents the number of positive samples in the determined anchor, that is, the number of subjects. The label information generates an embedding space, which is more compact than under self supervision, and helps the positive samples to have a tighter in distribution in the embedding space.

Furthermore, after contrastive learning, the encoder has learned to recognize the underlying logical features. The trained encoder is extracted and used for the next classification. The input will no longer be positive and negative sample pairs, but random and disordered test samples. The encoder parameters are determined by the previous stage, and in this stage, the encoder parameters are frozen and only pass through the classification head trained through the cross entropy loss function and composed of fully connected layers and activation functions.

The beneficial effects of the present disclosure are as follows. Experimental results show that the method provided by the present disclosure has higher recognition accuracy and smaller standard deviation compared with most advanced methods at present, and it can be seen that the performance of all the methods on the SEED dataset is superior to that of the SEED IV, that is because under the same experimental paradigm, the SEED IV dataset belongs to four classifications and has less data volume. Compared with other methods, especially on the SEED IV dataset with greater challenges, the results of the present disclosure have improved by at least 5% compared with the existing methods, which indicates that the method of the present disclosure is less affected by the recognition category and has better generalization ability. And the recognition of each emotion is analyzed in more detail through the confusion matrix, the model of the present disclosure has a better performance for the category with strong emotional performance, which is in line with neurocognitive research: that is, strong emotions have more obvious features and similarities than calm emotions. In the model of the present disclosure, the LSTM is used to capture the temporal feature correlations and predict the relevant emotion, so the length of the sample and the data volume will affect the effect of the experiment, so the number of samples is compared and analyzed, which shows that the optimal effect has achieved if six samples are used as a minibatch. Meanwhile, the proposed loss function (S-Info NCE) has also been conducted ablation analysis, and the loss function provided by the present disclosure can maximize both the correlation and difference among samples, so that the identification effect is better.

### BRIEF DESCRIPTION OF THE DRAWINGS

FIG. 1 is a model framework diagram of the present disclosure based on contrastive prediction (different shapes represent different subjects, and the same filling pattern represent the same emotion);

FIG. 2 is a framework diagram of a positive and negative sample design of the present disclosure;

FIG. 3 is a framework diagram of sample feature extraction of the present disclosure;

FIG. 4 is a structural diagram of the contrastive predictive coding model of the present disclosure (in which the parameters of the encoder g_{enc} are shared);

FIG. 5 is a framework diagram of classifier training of the present disclosure;

FIG. 6 is a schematic diagram of the experimental process of the SEED dataset of the present disclosure;

FIG. 7 is a contrastive chart of the performance differences of the methods of the present disclosure on two datasets; and

FIG. 8 is a confusion matrix diagram of various datasets under various emotions of the present disclosure.

### DETAILED DESCRIPTION OF THE EMBODIMENTS

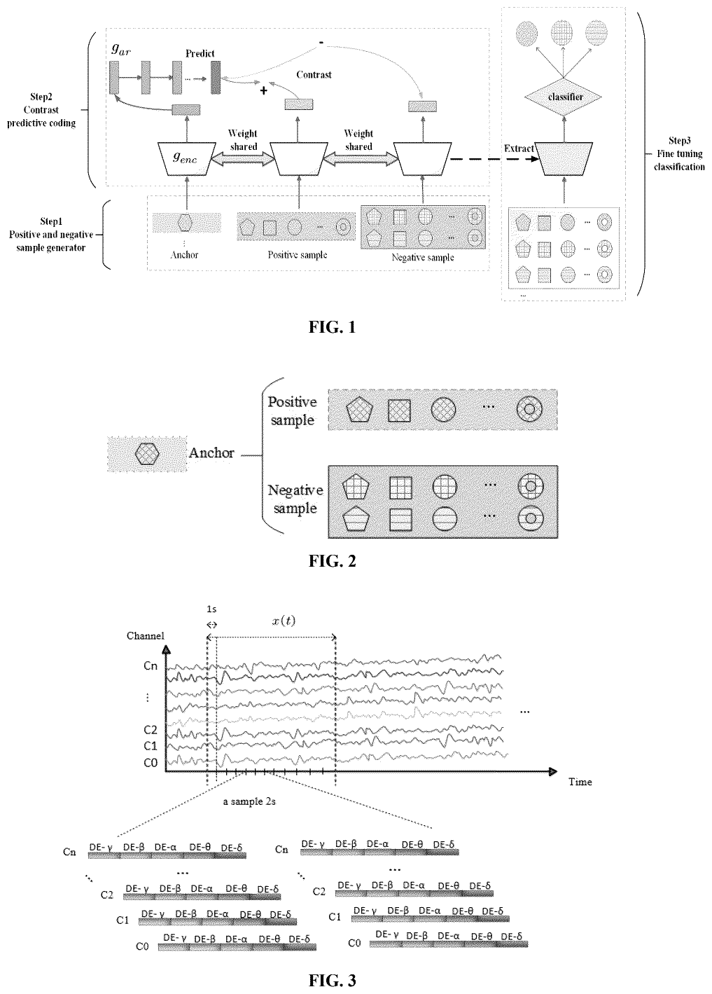

In order to make the technical methods adopted by the present disclosure and the purpose achieved easy to understand, a method of emotion recognition in cross-subject EEG signals is further described below in combination with specific embodiments. The electroencephalogram signal emotion recognition based on the contrastive predictive coding provided by the present disclosure consists of three parts, namely a positive and negative sample generator, a supervised contrastive coding representation and a fine tuning classification, as shown in FIG. 1. Specifically, firstly, the extracted Differential Entropy features are constructed as positive and negative samples using a positive and negative sample generator. Then, the DE features of an anchor and the positive and negative samples are fed into the encoder for coding, and mapped to the latent space; the encoded anchor samples are performed regression prediction in the latent space by using an autoregressive model. The encoder is trained using a supervised contrastive loss function, which narrows the distance between positive sample pairs and widens the distance between negative sample pairs to complete representation learning. After completing representation learning, the autoregressive model will be discarded. Finally, the trained encoder is connected to the classifier for fine tuning, and the classifier is trained using the cross entropy loss function. During this process, the encoder does not perform gradient propagation to complete cross-subject emotion recognition.

### 1. Positive and Negative Sample Generator

In the process of constructing positive and negative samples, the strategy of setting positive and negative samples with supervised contrastive loss is combined, and the label information of the samples is included in the design of positive and negative samples. The purpose of designing the positive and negative sample generator is to generate mini batches as inputs for the contrastive learning encoder. In the positive and negative samples constructed by the present disclosure, the strategy of setting positive and negative samples with supervised contrastive loss is combined to incorporate the label information of the samples into the design of the positive and negative samples. Defining that I={+,−,× . . . } represents the set of emotions, taking the SEED dataset as an example, representing three types of emotions respectively: happy, sad and neutral, S={1,2,3, . . . ,n} represents the set of n subjects, all samples can be marked as

$${X}_{q}^{k} , {X}_{q}^{k} ∈ H$$

(q_{∈S}, k_{∈I},

$${X}_{q}^{k} ∈ {R}^{{C}^{*} D} ,$$

C represents the number of channels, D represents the feature dimension extracted within a certain time, and H represents all sample sets under this dataset). In a batch, first determine the fixed emotion sample

$$f ∈ {X}_{1}^{+}$$

of the subject 1, and then take samples of subject 1 under the same emotion as positive samples in each experiment, that is,

$${f}^{+} ∈ {X}_{q}^{+} ( q ∈ S ) .$$

The number of f⁺ is n*p⁺(p⁺ represents the number of experimental segments in the dataset that evoke+emotions). Take all samples of subjects with different emotions from the positive sample in each experiment as negative samples, that is,

$$f ∈ {X}_{q}^{+} ( q ∈ S , k ∈ I , k \neq + ) ,$$

wherein the number of f is n*p^{k}(k_{∈I},k≠+). FIG. 2 shows the design of the positive and negative samples.

In order to fully capture the features of the samples in one batch, the mini batch will be extended by taking 6 consecutive sample sequences instead of taking one sample (one sample for 2 seconds) in each experiment. As shown in FIG. 3. Definition: In the process of a fixed subject conducting an experiment (that is, the emotion caused by a certain stimulus), such as in the SEED dataset, wherein the average duration of the stimulus is 4 minutes and there are 3 types of emotion classifications, 20 anchor samples will be generated, with 4*60/(2*6)=20 anchor samples. Each anchor corresponds to N positive samples and 2N negative samples, and their set e={f,f⁺,f} is used as a batch. In the next batch, the anchors and positive and negative samples are reselected until all samples are used as anchors, and then the training of an epoch is completed.

### 2. Supervised Contrastive Predictive Coding Representation

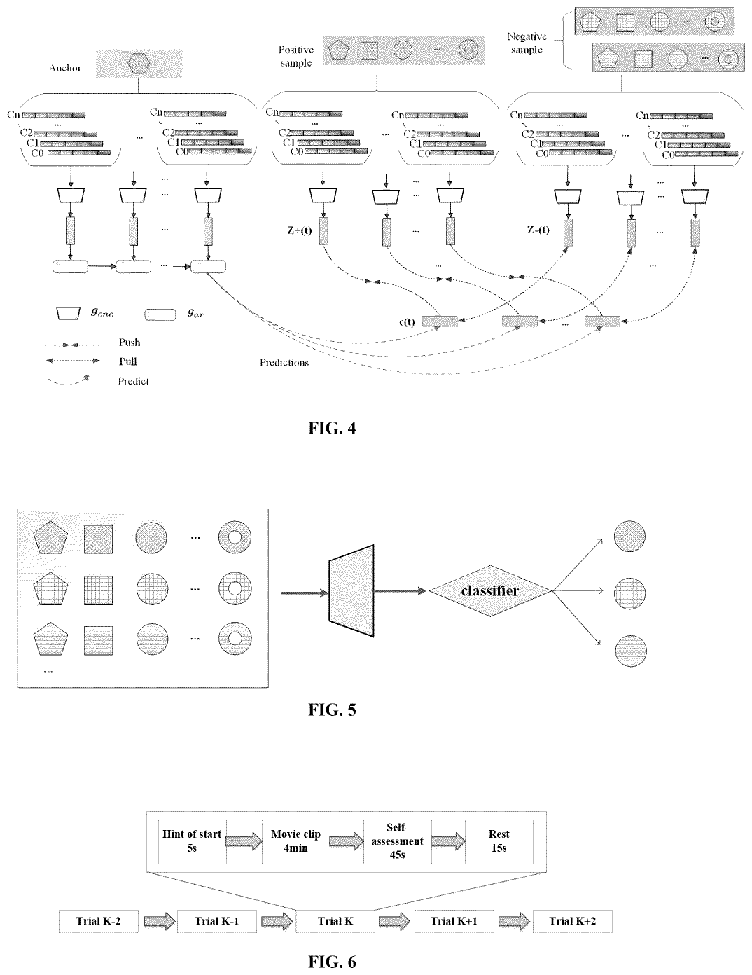

The purpose of contrastive predictive coding designs is to construct a feature extraction network that allows positive sample pairs to approach and negative sample pairs to move away. First, a nonlinear encoder g_{enc} maps an input sequence x(t) to a latent representation sequence z(t)=g_{enc}(x2), then an autoregressive model gₐᵣ summarizes all z≤t in the latent space and predicts a latent representation c(t)=gₐᵣ(z(t)). In contrastive predictive coding learning, since the EEG signal dataset belongs to a small-scale dataset, residual structures are used as the encoder g_{enc} to avoid overfitting, the anchor samples and the positive and negative samples enter the encoder in batches to obtain zₖ(t), the anchor samples enter an LSTM autoregressive model gₐᵣ to obtain a prediction result c(t). Considering that the features obtained by the anchor only through the encoder will have lower temporal resolution, LSTM is added as the the autoregressive model gₐᵣ. In the prediction process, the network learns the underlying features of the anchor emotions, so the prediction result c(t) obtained is a feature representation with the anchor emotions. The prediction result and the feature z₊(t) obtained by coding the positive sample form a positive sample pair; the prediction result and the feature z₋(t) formed with the negative sample coding is a negative sample pair; and finally the distance of the positive sample pair is narrowed and the distance of the negative sample pair is widened through the supervised contrastive loss function to complete the contrastive predictive coding. The framework of the supervised contrastive predictive coding is shown in FIG. 4, taking 1 second per sample as an example.

The basic idea of the NCE loss function is to distinguish between correct samples and a set of noisy samples, and the model is trained by maximizing the probability of the correct samples and minimizing the probability of the noise samples; in contrastive learning, the model is trained by comparing the positive sample and the negative sample, as shown in formula (1):

$${L}_{p} = - log \frac{exp ( m \cdot {p}_{+} / τ )}{{\sum }_{i = 0}^{K} exp ( m \cdot {p}_{i} / τ )} \qquad ( 1 )$$

Wherein, m [symbol] p is the representation vector obtained by the sample passing through the f(·) network, m·p₊ is the dot product similarity between the anchor and the positive sample, m·pᵢ is the dot product similarity between the anchor and other samples, K represents the number of negative samples, and r is a temperature parameter.

In combination with the idea of CPC, the training of both the encoder and the the autoregressive model gₐᵣ is also included in this loss function, and both the encoder and the the autoregressive model gₐᵣ are trained to jointly optimize the loss based on NCE, as shown in formula (2):

$${L}_{N} = - log \frac{exp ( {c}_{h} \cdot {z}_{q} / τ )}{{\sum }_{a □ A ( h )} exp ( {c}_{h} \cdot {z}_{a} / τ )} \qquad ( 2 )$$

Wherein, cₕ is the predicted vector of the anchor sample h obtained through gₐᵣ(g_{enc}(xₜ)), zₐ is the representation vector of the positive sample of the subject q obtained through g_{enc}(xₜ), A(h)=e\h, zₐ are the representation vectors of samples other than anchors in a batch obtained through g_{enc}(xₜ). Unlike CPC, which only considers samples from anchors as positive samples, due to the presence of label information, using label information combined with CPC loss, each anchor can have multiple positive samples, that is, samples with the same label are positive samples, making contrastive learning suitable for fully supervised situations, as shown in the following formula (3):

$${L}^{sup} = {\sum }_{h □ H} {L}_{h} = {\sum }_{h □ H} \frac{- 1}{\vert q ( h ) \vert} {\sum }_{q □ S} log \frac{exp ( {c}_{h} \cdot {z}_{q} / τ )}{{\sum }_{a □ A ( h )} exp ( {c}_{h} \cdot {z}_{a} / τ )} \qquad ( 3 )$$

q(h) represents the number of positive samples in the determined anchor, that is, the number of subjects. The label information generates an embedding space, which is more compact than under self supervision, and helps the positive samples to have a tighter distribution in the embedding space.

### 3. Fine Tuning Classification

After contrastive learning, the encoder has learned to recognize the underlying logical features. The trained encoder is extracted and used for the next classification. The classifier structure is shown in FIG. 5. At this point, the input will no longer be positive and negative sample pairs, but random and disordered test samples. The encoder parameters are determined by the previous stage, and in this stage, the encoder parameters are frozen and only pass through the classification head trained through the cross entropy loss function and composed of fully connected layers and activation functions.

### Experiment

In the present disclosure, SEED and SEED IV are used as training datasets. Firstly, the dataset and preprocessing process are introduced. Secondly, the experimental design scheme and implementation details are presented. Finally, the experimental results are presented and discussed.

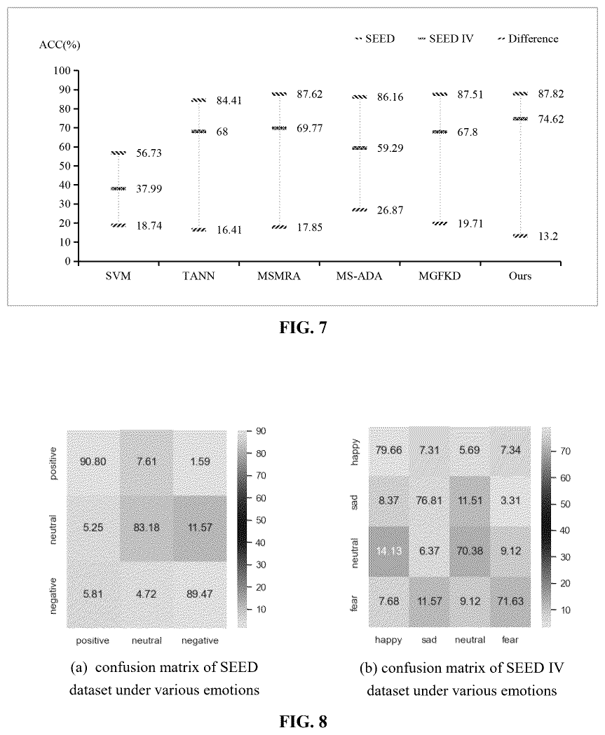

The SEED dataset is a publicly available EEG signal dataset widely used for emotion recognition. This dataset includes EEG data from 15 subjects (8 females, with an average age of 23.27 years and an age standard deviation of 2.37 years). Each subject was required to conduct three experiments at different times, with an interval of more than one week between sessions. The experimental team prepared movie clips that could evoke positive emotions, neutral emotions and negative emotions, five clips for each emotion, with an average duration of 226 seconds, a total of 15 movie clips. The subjects watched these clips in each session. The experimental process included 5 seconds of prompt time, 4 minutes of movie clips, 45 seconds of self-assessment time and 15 seconds of rest time. As shown in FIG. 6, the corresponding emotions are stimulated by immersive viewing.

The experimental team used a 10-20 system to collect 62 channels of EEG signals with a sampling frequency of 1000 Hz. The publicly available SEED dataset has downsampled the data to 200 Hz and filtered it from 0 to 75 Hz. At the same time, the publicly available dataset also provides preprocessed experimental data. Differential Entropy (DE) features further smoothed by the linear dynamic system (LDS) method are selected as experimental samples, and non-overlapping time windows of one second are taken as a sample, and five frequency bands (δ:1-3 Hz, θ:4-7 Hz, α: 8-13 Hz, β:14-30 Hz, γ: 31-50 Hz) of EEG signals are extracted from each sample to calculate their DE features. Previous studies have shown that differential entropy (DE) features have stronger discriminant ability in emotion recognition. In order to provide more stable DE features to reduce the influence of noise randomness and ensure the number of samples, 62 DE values in 5 frequency bands are concatenated. In this paper, consistent with most literatures, adjacent 2s DE features are selected for splicing to obtain samples with feature vector dimension of 62*10. In order to ensure the tractability and consistency of all datasets, the inputs are uniformly populated as 64*10.

The SEED IV dataset is also a widely used EEG signal dataset for emotion recognition. This dataset includes EEG signals from 15 subjects. In each session, movie clips were set to evoke four emotions: happiness, sadness, neutrality, and fear, and each emotion corresponds to four different clips, and each subject watched 24 movie clips in each session.

The preprocessing methods provided by the disclosed SEED-IV dataset and the SEED dataset are the same, and the same preprocessing method is still selected in the present disclosure. The only difference is that the SEED IV dataset takes a non-overlapping 4-second time window as a sample to calculate the DE features of different frequency bands. Compared with the SEED dataset, the DE features extracted from the SEED-IV dataset include sufficient time features, and in order to ensure sufficient data volume to train the model, the input is not spliced. The feature dimension of one sample is 62*5, which is filled with 64*10. Table 1 shows the description of feature size and feature dimension of the two datasets.

TABLE 1
Dataset description

| Size | SEED | SEED-IV |
|---|---|---|
| Sample Size of Feature/ | 3*15*(3394/2) = 76365 | 3*15*851 = 38295 |
| Sample Size of Label | session*subject*(trial/2s) | session*subject*trial |
| Feature Dimension | 64*10 | 64*10 |

In the cross-subject experiment, a leave-one-out experiment of the cross-subject is conducted on the SEED and SEED IV datasets respectively. In order to compare with the most advanced experiment results, the present disclosure is consistent with the experimental setup of the most advanced experiments. Specifically, the test set of the experiment in the present disclosure is a sample of a certain subject, and the training set and the verification set are all samples except the test subjects. If there are N samples in the dataset, N times of leave-one-out experiments are required. In order to verify the rationality of the parameter design of the framework proposed in the present disclosure, a comparative experiment is designed. In the designed framework, the number of samples in each minibatch will affect the effect of the experiment. In a minibatch, too many samples will lead to insufficient total training data volume, and too few samples are not conducive to capturing the potential emotional features of the network, so the number of samples and the total of training data volume should be considered comprehensively. Experiments have shown that about 10s can characterize a person's emotions. In the SEED dataset, the length of a sample is 2s. Therefore, the number of positive (negative) samples in a batch is designed to be 4, 5, 6, 7 and 8 respectively to find the optimal number suitable for the dataset of the present disclosure. Meanwhile, in order to verify the effectiveness of the loss function, an ablation experiment is performed on the loss function mentioned in the present disclosure.

The model training of the present disclosure uses the Python 3.8 programming language and the keras development framework to implement the model. For the encoder, a network model with a 16-layer residual structure is designed according to the dimension of the input data and the size of the dataset. The parameters of the network model are shown in the Table 2. In each residual block, the residual connects two convolutional layers. It should be noted that in Resnet_layer2, Resnet_layer3, and Resnet_layer_4, the first layers of the residual module need to go through a convolution layer with a size of 1×1, M to increase the dimension, wherein M is the same as the number of channels for the residual structure output F(X) of this layer. A downsampling operation with a step size of 2 is required so that the residual F(X) and the identity map X can be added. The hidden dimension of the regression model LSTM is set to 128, the learning rate is set to 0.001 with Adam as the optimizer, and the learning rate is halved if the loss is not reduced for three consecutive times. The batch size is 64, and 80 epochs are trained in the first coding prediction phase and 40 epochs in the second classification phase.

TABLE 2
Encoder network parameter structure

| Layer name | Input size | Layer construction | Output size |
|---|---|---|---|
| Conv1 | 64 × 10, 1 | 3 × 3, 32 | 64 × 10, 32 |
| | | Stride 1, padding 1 | |
| Resnet_layer1 | 64 × 10, 32 | $[ \begin{matrix}3 \times 3 , & 32 \\ 3 \times 3 , & 32\end{matrix} ] \times 2$ | 64 × 10, 32 |
| Resnet_layer2 | 64 × 10, 32 | $[ \begin{matrix}3 \times 3 , & 64 \\ 3 \times 3 , & 64\end{matrix} ] \times 2$ | 32 × 5, 64 |
| Resnet_layer3 | 32 × 5, 64 | $[ \begin{matrix}3 \times 3 , & 128 \\ 3 \times 3 , & 128\end{matrix} ] \times 2$ | 16 × 3, 128 |
| Resnet_layer4 | 16 × 3, 128 | $[ \begin{matrix}3 \times 3 , & 256 \\ 3 \times 3 , & 256\end{matrix} ] \times 2$ | 8 × 2, 256 |
| Max Pooling, FC | 8 × 2, 256 | — | 256 |

In order to prove the effectiveness of the present disclosure, the results of the cross-subject experiments on the SEED dataset were compared with six specific methods of the following various methods: Support Vector Machines (SVM), novel Transferability Attention Neural Network (TANN), Contrastive Learning Inter-Subject Alignment (CLISA), Multi-origin and Multi-presentation Adjustment (MSMRA), Multisource Associate Domain Adaptation (MS-ADA) and Multi-domain Geodesic Flow Kernel Dynamic Distribution Alignment (MGFKD). Table 3 lists the accuracy of some methods in the SEED dataset for each subject (the data listed in Table 3 are derived from the accuracy of each subject's method given in the corresponding paper). Table 4 and Table 5 list the mean precision and standard deviation of the across-subjects on the SEED and SEED IV datasets.

TABLE 3
Accuracy (%) of various methods across-subjects in SEED dataset

| Sub | CLISA | MGFKD | OURS | Sub | CLISA | MGFKD | OURS |
|---|---|---|---|---|---|---|---|
| 1 | 75.5 | 84.9 | 89.7 | 9 | 90.8 | 92.3 | 90.0 |
| 2 | 82.2 | 70.9 | 86.0 | 10 | 83.4 | 81.6 | 78.9 |
| 3 | 87.6 | 80.4 | 78.7 | 11 | 91.7 | 88.8 | 83.9 |
| 4 | 85.8 | 92.1 | 86.8 | 12 | 69.8 | 81.2 | 85.9 |
| 5 | 86.0 | 87.7 | 89.4 | 13 | 91.7 | 86.3 | 92.1 |
| 6 | 86.6 | 100.0 | 95.8 | 14 | 90.3 | 90.1 | 85.7 |
| 7 | 88.6 | 84.1 | 87.7 | 15 | 93.0 | 100.0 | 92.5 |
| 8 | 93.2 | 83.9 | 93.6 | Avg | 86.4 | 86.9 | 87.8 |

TABLE 4
Average accuracy and standard deviation of various
methods across-subjects in SEED dataset

| Method | SVM | TANN | CLISA | MSMRA | MS-ADA | MGFKD | Ours |
|---|---|---|---|---|---|---|---|
| Year | 1999 | 2021 | 2022 | 2022 | 2023 | 2024 | 2024 |
| ACC(%) | 56.73 | 84.41 | 86.40 | 87.62 | 86.16 | 86.93 | 87.82 |
| STD(%) | 16.29 | 8.75 | 6.40 | 7.53 | 7.87 | 7.28 | 5.05 |

TABLE 5
Average accuracy and standard deviation of various
methods across-subjects in SEED IV dataset

| Method | SVM | TANN | CLISA | MSMRA | MS-ADA | MGFKD | Ours |
|---|---|---|---|---|---|---|---|
| Year | 1999 | 2021 | 2022 | 2022 | 2023 | 2024 | 2024 |
| ACC(%) | 37.99 | 68.00 | — | 69.77 | 59.29 | 67.80 | 74.62 |
| STD(%) | 12.52 | 8.35 | — | 7.37 | 13.65 | 8.25 | 5.31 |

In Table 3, it can be seen that the accuracy of more subjects with the method of the present disclosure is higher than that of the other two methods. Due to the difference of models, the latent space found by the features extracted by the models is also different, so the performance of subjects on different models is not consistent. In Table 4 and Table 5, it can be seen that the average accuracy of the present disclosure on the SEED dataset is 87.82%, and the standard deviation is 5.05%; the average accuracy of the present disclosure on the SEED IV dataset is 74.62%, and the standard deviation is 5.31%. It is better than the machine learning, domain adaptation, contrastive learning, domain transfer, semi-supervision and other methods listed in the present disclosure, and the standard deviation is also lower. Compared with the domain adaptation method MS-ADA which needs to access the test set in the network training process, the method of the present disclosure improves about 1.6% on the SEED dataset and about 15% on the SEED IV dataset. Compared with CLISA using contrastive learning, the accuracy of the supervised prediction contrastive learning used in the present disclosure is also improved by about 1.4%, and obtained consistent conclusions with CLISA, while the number of categories in the dataset had a smaller impact on the model. Therefore, compared with the advanced cross-subject method in recent years, the model provided by the disclosure has higher recognition accuracy.

Especially on the SEED IV dataset, the accuracy of the method of the present disclosure exceeds the suboptimal accuracy by nearly 5%. FIG. 7 shows the performance difference of each method on the two datasets. Compared with the SEED three classification set, SEED IV has one more category, which may be the reason for the decrease in the accuracy of all methods. However, due to the excellent performance of contrastive learning in multi-category tasks, this can explain that the impact of the number of categories on the model of the present disclosure is lower than that of other methods. In FIG. 7, the section lines inclined to the left represent the data points of the SEED dataset, the grid lines represent the data points of the SEED IV dataset, and the section lines inclined to the right represent the difference values of the data points in the SEED dataset and the data points in the SEED IV dataset under each emotion.

In addition, the present disclosure also analyzes the recognition rate of each emotion in the form of a confusion matrix. FIG. 8 shows the confusion matrix of the method of the present disclosure in the SEED dataset and the SEED IV dataset, from which the average recognition accuracy under each emotion can be obtained. It can be seen that in the SEED dataset, the recognition accuracy of positive emotions is up to 90.8%, and 7.61% and 1.59% of positive emotions are misrecognized as neutral and negative. In the SEED dataset, the model has a strong effect on the classification of positive and negative emotions, but a weak effect on the classification of neutral emotions. This phenomenon can be attributed to the complexity of neutral emotions, which are not as strong as positive emotions and negative emotions, and do not have too obvious neural patterns. In the SEED IV dataset, the recognition effect of happy emotions and sad emotions is also better than that of neutral emotions.

At the same time, the number of samples in minibatch is compared. In the SEED dataset, subject 2, subject 6 and subject 9 were randomly selected, and different sample numbers 4, 5, 6, 7 and 8 were set to find the optimal sample number. As shown in Table 6, the three subjects all have the best recognition accuracy if the number of samples is 6, which indicates that the appropriate number of samples and sufficient data volume are helpful for the network to capture signal features and improve the recognition accuracy.

TABLE 6
Identification results of different
sample sizes of random subjects (%)

| Size | Sub_2 | Sub_6 | Sub_9 |
|---|---|---|---|
| 4 | 81.98 | 92.18 | 83.17 |
| 5 | 84.88 | 94.56 | 87.60 |
| 6 | 86.00 | 95.81 | 89.98 |
| 7 | 84.12 | 93.46 | 88.25 |
| 8 | 82.60 | 90.79 | 86.74 |

To verify the effectiveness of the Supervised-Info Noise Contrastive Estimation (S-info NCE) Loss Function, ablation experiments were also conducted for the loss function in the contrastive prediction learning phase. Subject 2, subject 6 and subject 9 were randomly selected to calculate their average accuracy under the two loss functions, the S-info NCE loss function has higher accuracy compared to the basic cross entropy loss function, as shown in Table 7. This shows that the S-info NCE loss function can promote the diversity and discrimination of feature learning. By maximizing mutual information, it encourages the model to learn to capture correlations and differences between samples, resulting in more informative feature representations and improving the accuracy of cross-subject identification.

TABLE 7
Identification results of different loss functions

| Loss | Acc (%) |
|---|---|
| S-Info NCE | 90.60 |
| Cross-Entropy | 87.46 |

Experimental results show that the method provided by the present disclosure has higher recognition accuracy and smaller standard deviation compared with most advanced methods at present, and it can be seen that the performance of all the methods on the SEED dataset is superior to that of the SEED IV, that is because under the same experimental paradigm, the SEED IV dataset belongs to four classifications and has less data volume. Compared with other methods, especially on the SEED IV dataset with greater challenges, the result of the present disclosure have improved by at least 5% compared with the existing methods, which indicates that the method of the present disclosure is less affected by the recognition category and has better generalization ability. And the recognition of each emotion is analyzed in more detail through the confusion matrix, the model of the present disclosure has a better performance for the category with strong emotional performance, which is in line with neurocognitive research: that is, strong emotions have more obvious features and similarities than calm emotions. In the model of the present disclosure, the LSTM is used to capture the temporal feature correlations and predict the relevant emotion, the length of the sample and the data volume will affect the effect of the experiment, so the number of samples is compared and analyzed, which shows that the optimal effect has achieved if six samples are used as a minibatch. Meanwhile, the proposed loss function (S-Info NCE) has also been conducted ablation analysis, and the loss function provided by the present disclosure can maximize both the correlation and difference among samples, so that the identification effect is better.

The above description is only a preferred specific embodiment of the present disclosure, but the scope of protection of the present disclosure is not limited to this. Any skilled person familiar with the technical field should be included in the scope of protection of the present disclosure by equivalent substitution or modification based on the technical solution and concept disclosed in the present disclosure.

What is claimed is:

1. A method of emotion recognition in cross-subject EEG signals, comprising:
S1, constructing extracted Differential Entropy features into positive and negative samples by utilizing a positive and negative sample generator;
S2, sending the Differential Entropy features of an anchor and the positive and negative samples into an encoder for coding, mapping the Differential Entropy features of the anchor and the positive and negative samples to a latent space, performing regression prediction on encoded anchor samples in the latent space by utilizing an autoregressive model, training the encoder by utilizing a supervision contrastive loss function, training the encoder to complete representation learning by narrowing a distance between positive sample pairs and widening a distance between negative sample pairs, and discarding the autoregressive model after the representation learning is completed; and
S3, connecting the trained encoder to a classifier for fine tuning, and training the classifier through a cross entropy loss function; in this process, the encoder does not perform gradient propagation to complete cross-subject emotion recognition.

2. The method of emotion recognition in cross-subject EEG signals according to claim 1, wherein in the constructed positive and negative samples, a strategy is set by combining the positive and negative samples of supervised contrastive loss, label information of the samples is comprised in a design of the positive and negative samples, and a mini batch generated by the positive and negative sample generator is utilized as an input of a contrastive learning encoder; define that I={+,−,× . . . } represents a set of emotions, in a SEED dataset, representing three types of emotions respectively: happy, sad and neutral, S={1,2,3, . . . ,n} represents a set of n subjects, all samples can be marked as
$${X}_{q}^{k} , {X}_{q}^{k} ∈ H ,$$
q_{∈S}, K_{∈I},
$${X}_{q}^{k} ∈ {R}^{C * D} ,$$
represents a number of channels, D represents feature dimension extracted within a certain time, and H represents all sample sets under this dataset;
in a batch, first a fixed emotion sample
$$f ∈ {X}_{1}^{+}$$
of subject 1 is determined, and then samples of the subject 1 under same emotion are taken as positive samples in each experiment, that is,
$${f}^{+} ∈ {X}_{q}^{+} ( q ∈ S ) ;$$
a number of f⁺ is n*p⁺, and p⁺ represents a number of experimental segments in a dataset that evoke+emotions; all samples of subjects with different emotions are taken from the positive sample in each experiment as negative samples, that is,
$$f ∈ {X}_{q}^{+} ( q ∈ S , k ∈ I , k \neq + ) ,$$
wherein a number of f is n*p^{k}(k_{∈I},k≠+);
in order to fully capture features of samples in a batch, the mini batch is extended by taking 6 consecutive sample sequences for 2 seconds per sample; definition: in a process of a fixed subject conducting an experiment, that is, the emotion caused by a certain stimulus, in the SEED dataset, wherein an average duration of the stimulus is 4 minutes and there are 3 types of emotion classifications, 20 anchor samples will be generated, with 4*60/(2*6)=20 anchor samples; each anchor corresponds to N positive samples and 2N negative samples, and their set e={f,f⁺,f} is utilized as a batch; in a next batch, the anchors and the positive and negative samples are reselected until all samples are utilized as anchors, and then a training of an epoch is completed.

3. The method of emotion recognition in cross-subject EEG signals according to claim 2, wherein a feature extraction network is constructed by a contrastive predictive coding design, so that the positive sample pairs are close to each other and the negative sample pairs are far away from each other;
first, a nonlinear encoder g_{enc} maps an input sequence x(t) to a latent representation sequence z(t)=g_{enc}(xₜ), and an autoregressive model gₐᵣ summarizes all z≤t in the latent space and predicts a latent representation c(t)=gₐᵣ(z(t)); in contrastive predictive coding learning, a residual structure is utilized as the encoder gₐᵣ to avoid over-fitting, the anchor samples and the positive and negative samples enter the encoder in batches to obtain zₖ(t), the anchor samples enter an LSTM autoregressive model gₐᵣ to obtain a prediction result c(t); the LSTM is added as the autoregressive model gₐᵣ to improve time resolution of features; in the prediction process, the network learns underlying features underlying the anchor emotions, the prediction result c(t) is a feature representation with a fixed point emotion; the prediction result and a feature z₊(t) obtained by coding the positive sample form a positive sample pair;
the prediction result and a feature z₋(t) formed with the negative sample coding is a negative sample pair; and finally the distance of the positive sample pair is narrowed and the distance of the negative sample pair is widened through the supervised contrastive loss function to complete the contrastive predictive coding;
a correct sample is distinguished from a set of noise samples by the Noise Contrastive Estimation (NCE) loss function, and the model is trained by maximizing probability of the correct sample and minimizing probability of the noise sample; in contrastive learning, the model is trained by comparing the positive sample and the negative sample, as shown in formula (1):
$${L}_{p} = - log \frac{exp ( m \cdot {p}_{+} / τ )}{{\sum }_{i = 0}^{K} exp ( m \cdot {p}_{i} / τ )} \qquad ( 1 )$$
wherein, m·p is a representation vector obtained by the sample passing through the network f(·), m·p₊ is dot product similarity between the anchor and the positive sample, m·pᵢ is dot product similarity between the anchor and other samples, K represents a number of negative samples, and τ is a temperature parameter;
in combination with an idea of contrastive predictive coding, a training of both the encoder and the autoregressive model gₐᵣ is also compried in this loss function, and both the encoder and the autoregressive model gₐᵣ are trained to jointly optimize loss based on NCE, as shown in formula (2):
$${L}_{N} = - log \frac{exp ( {c}_{h} \cdot {z}_{q} / τ )}{{\sum }_{a □ A ( h )} exp ( {c}_{h} \cdot {z}_{a} / τ )} \qquad ( 2 )$$
wherein, cₕ is a predicted vector of an anchor sample h obtained through gₐᵣ(g_{enc}(xₜ)), z_{q} is a representation vector of a positive sample of the subject q obtained through g_{enc}(xₜ), A(h)=e\h, zₐ are representation vectors of samples other than anchors in a batch obtained through g_{enc}(xₜ); unlike CPC, which only considers samples from anchors as positive samples, using label information combined with CPC loss, each fixed point can have multiple positive samples, that is, samples with a same label are positive samples, making contrastive learning suitable for fully supervised situations, as shown in the following formula (3):
$${L}^{sup} = {\sum }_{h □ H} {L}_{h} = {\sum }_{h □ H} \frac{- 1}{\vert q ( h ) \vert} {\sum }_{q □ S} log \frac{exp ( {c}_{h} \cdot {z}_{q} / τ )}{{\sum }_{a □ A ( h )} exp ( {c}_{h} \cdot {z}_{a} / τ )} \qquad ( 3 )$$
wherein, q(h) represents a number of positive samples in a determined anchor, that is, a number of subjects; the label information generates an embedding space, which is more compact than under self supervision, and helps the positive samples to have a tighter distribution in the embedding space.

4. The method of emotion recognition in cross-subject EEG signals according to claim 3, wherein after the contrastive learning, the encoder has learned to recognize underlying logical features; the trained encoder is extracted and utilized for a next classification; the input of the trained encoder will no longer be positive and negative sample pairs, but random and disordered test samples; encoder parameters are determined by a previous stage, and in this stage, the encoder parameters are frozen and only pass through a classification head trained through a cross entropy loss function and composed of fully connected layers and activation functions.
